(19) United States
(12) Patent Application Publication
(10) Pub. No.: US 2025/0257372 A1
(43) Pub. Date: Aug. 14, 2025

(54) USE OF CYANOBACTERIAL BIOREACTOR FOR CARBON SEQUESTRATION

(71) Applicant: BIODEL AG INC., MARICOPA, AZ (US)

(72) Inventors: GEORGE BENJAMIN CLOUD, MARICOPA, AZ (US); MICHAEL KIRKSEY, MARICOPA, AZ (US); KENT ADAMS, ORO VALLEY, AZ (US); PAUL ZORNER, ENCINITAS, CA (US)

(21) Appl. No.: 18/856,332

(22) PCT Filed: Apr. 7, 2023
(86) PCT No.: PCT/US2023/065505

§ 371 Date: Oct. 11, 2024

Related U.S. Application Data
(60) Provisional application No. 63/329,705, filed on Apr. 11, 2022.

(51) Int. Cl. C12P 7/24

(52) U.S. Cl. CPC C12P 7/24

(57) ABSTRACT

The subject invention provides compositions and methods for reducing deleterious atmospheric gases using microorganisms. In preferred embodiments, a composition comprising one or more beneficial microorganisms convert carbon dioxide to calcium carbonate, magnesium carbonate, a bicarbonate, or a combination thereof. The source of the carbon dioxide can be industrial emissions. In some embodiments, the composition sequesters carbon with the use of a trough growth system and the culture and/or growth by-product can be sequestered in underground geological formations.

### CROSS-REFERENCE TO RELATED APPLICATION

This application claims priority to U.S. Provisional Patent Application No. 63/329,705, filed Apr. 11, 2022, which is incorporated herein by reference in its entirety.

### BACKGROUND OF THE INVENTION

“Greenhouse gases,” or “GHG,” include carbon dioxide, methane, nitrous oxide, and fluorinated gases that trap heat in the atmosphere. Carbon dioxide enters the atmosphere through, for example, burning fossil fuels (coal, natural gas, and oil), solid waste, trees and wood products, and also as a result of certain chemical reactions, including through the manufacturing of cement. Carbon dioxide is removed from the atmosphere by, for example, absorption by plants and other photosynthetic organisms as part of the biological carbon cycle. Based on recent measurements from monitoring stations around the world and measurement of older air from air bubbles trapped in layers of ice from Antarctica and Greenland, global atmospheric concentrations of GHGs have risen significantly over the last few hundred years (EPA report 2016 at, e.g., 6, 15).

Especially since the Industrial Revolution began in the 1700s, human activity has contributed to the amount of GHGs in the atmosphere by burning fossil fuels, cutting down forests, overly cultivating soils, and conducting other activities. Many GHGs emitted into the atmosphere remain there for long periods of time ranging from a decade to many millennia. Over time these gases are removed from the atmosphere by chemical reactions or by emissions sinks, such as the oceans, vegetation, and soils that absorb GHGs from the atmosphere.

World leaders have attempted to curb the increase of GHG emissions through treaties and other inter-state agreements. One such attempt is through the use of carbon credit systems. A carbon credit is a generic term for a tradable certificate or permit representing the right to emit one ton of carbon dioxide, or an equivalent GHG. In a typical carbon credit system, a governing body sets quotas on the amount of GHG emissions an operator can produce. Exceeding these quotas requires the operator to purchase extra allowances from other operators who have not used all of their carbon credits.

One goal of carbon credit systems is to encourage companies to invest in more green technology, machinery and practices in order to benefit from the trade of these credits. Under the Kyoto Protocol of the United Nations Framework Convention On Climate Change (UNFCCC), a large number of countries have agreed to be bound internationally by policies for GHG reduction, including through trade of emissions credits. While the United States is not bound by the Kyoto Protocol, and while there is no central national emissions trading system in the U.S., some states, including California and a group of northeastern states, have begun to adopt such trading schemes.

There remain growing concerns over climate change and a need for both reducing GHG emissions and facilitating drawdown using improved approaches for carbon sequestration.

### BRIEF SUMMARY OF THE INVENTION

The subject invention provides compositions, methods, and systems for reducing atmospheric greenhouse gas emissions using beneficial microorganisms. More specifically, the subject invention provides multi-purpose compositions that, when grown, lead to a reduction in greenhouse gases in the atmosphere.

In specific embodiments, the subject invention provides a microbe-based composition for reducing atmospheric GHGs, wherein the composition comprises one or more beneficial microorganisms. In preferred embodiments, the beneficial microorganisms are cyanobacteria and/or algae. Advantageously, in preferred embodiments, the subject invention utilizes non-GMO microorganisms. In certain embodiments, a single microorganism strain can be used, or mixotrophic cultures can be used.

In one embodiment, the compositions help to reduce GHG concentrations in the atmosphere by converting carbon dioxide in the atmosphere to calcium carbonate or other mineral precipitates, including, for example, magnesium carbonate or a bicarbonate, such as, for example, sodium bicarbonate. Furthermore, the compositions can result in the production of algae- and cyanobacterial-based soil amendments, feed for livestock and aquaculture, and nutraceuticals.

In certain embodiments, the microorganism is a cyanobacterium, selected from, for example, Synechocystis, Synechococcus, Anabaena, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, Tolypothrix, and Scytonema.

In certain embodiments, the microorganism is an alga selected from, for example, Chlorella, Chlamydomonas, Dunaliella, Bracteacoccus, Prasinoderma, and Nannochloropsis.

In certain embodiments, the methods can also be used for enhancing carbon sequestration, wherein the mineral precipitates and, optionally, one or more beneficial microorganisms can be injected into a repository, such as, for example, a surface repository, including soil of a field, forest, prairie or pasture, or a sea, lake, or ocean floor; or an underground repository, including, for example, an underground geological formation, mature or depleted oil and gas reservoir, mine, cave, cavern, basalt formation, shale formation, deep saline formation, or unmineable coal scams. In certain embodiments, the mineral precipitates can enhance the amount of carbon sequestered in the pasture or field, thereby transforming the field or pasture into a carbon sink. The composition can be applied either as a liquid or a dried product. In one embodiment the composition is broadcast, either in the liquid or dried form, over the field or pasture using, for example, an irrigation system. Additionally, the composition can be applied using a manual spreader, such as a broadcast spreader, a drop spreader, a handheld spreader, or a handheld sprayer. In one embodiment, the composition is applied either in the liquid or dried form, to the surface or underground repository.

The methods can be used to sequester carbon released by industrial effluents and/or emissions. Sources of emissions or effluents include, for example, power generation stations, particularly those that generate electricity using coal, oil, natural gas, methane, or a combination thereof; industrial production plants, such as, iron and steel production plants, automobile manufacturing plants, petroleum refineries, cement production plants, glass production plants, food production plants, and ethanol or other biofuel production plants; and landfills. In certain embodiments, the methods can comprise the use of existing CO₂ scrubber systems for removal and/or storage of carbon dioxide from emission sources. The scrubbed CO₂ can be used as a carbon source for the growth of microorganisms. In certain embodiments, the methods can be used in conjunction with scrubber systems, either before the emissions are scrubbed, after the emissions are scrubbed, or both before and after the emissions are scrubbed. In some embodiments, the methods of the subject invention can be utilized for reducing carbon credit usage or for selling carbon credits. Thus, in certain embodiments, the subject methods can further comprise conducting measurements to assess the effect of the method on the sequestration of carbon in the repository, using standard techniques in the art.

In certain embodiments, the cyanobacteria and/or algae can be grown in a trough production system. In certain embodiments, the trough can be in the shape of a “V” as opposed to common flat-bottomed raceway growth systems. In certain embodiments, the trough can contain a nutrient/aeration rack that can provide mixing action and uniform nutrient distribution. The rack can be designed to be removable yet be of sufficient weight to stay on the bottom of the trough. In certain embodiments, the nutrient/aeration rack can combine aeration and motive force to the cyanobacteria and/or algae growing within the trough production system.

### BRIEF DESCRIPTION OF THE FIGURES

FIG. 1 shows the cross section of the trough system used for growth of algae and cyanobacteria.

FIG. 2 shows the nutrient/aeration rack of the trough system.

FIG. 3 shows the overhead view of the trough system used for growth of algae and cyanobacteria.

FIG. 4 shows the sloped screen concentrator for harvest.

FIG. 5 shows the overhead view of the trough system used for growth of algae and cyanobacteria.

FIG. 6 shows the aeration system of the trough system used for growth of algae and cyanobacteria.

FIG. 7 shows the nutrient lines of the trough system used for growth of algae and cyanobacteria.

FIG. 8 shows the nutrient lines of the trough system used for growth of algae and cyanobacteria.

### DETAILED DESCRIPTION OF THE INVENTION

The subject invention provides compositions and methods for reducing atmospheric greenhouse gas emissions using beneficial microorganisms. More specifically, the subject invention provides compositions comprising beneficial microorganisms that can convert GHGs to mineral precipitates. Furthermore, the production of mineral compounds can be a means of sequestering carbon. In certain embodiments, the mineral compounds produced by the microorganisms can also be used in nutraceuticals, animal feed, and soil amendments.

### Selected Definitions

As used herein, “harvested” refers to removing some or all of a microbe-based composition from a growth vessel.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, and surfactants.

As used herein, a “broth” or “culture broth,” or refers to a culture medium comprising at least nutrients and microorganism cells.

As used herein, a “biologically pure culture” is a culture that has been isolated from materials with which it is associated in nature. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “enhancing” means improving or increasing. For example, enhanced carbon sequestration means increasing the amount of atmospheric carbon sequestered in or on the Earth.

The subject invention utilizes “microbe-based compositions,” meaning a composition that comprises components that were produced as the result of the growth of microorganisms. Thus, the microbe-based composition may comprise the microbes themselves and, optionally, by-products of microbial growth. The microbes may be in a vegetative state, in spore (e.g., akinetes) heterocysts, or a mixture thereof. The microbes may be planktonic, in a biofilm form, or a mixture thereof. The by-products of growth may be, for example, metabolites, cell membrane components, proteins, and/or other cellular components. The microbes may be intact or lysed. In preferred embodiments, the microbes are present with growth medium in which they were grown in the microbe-based composition. The microbes may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or per ml of the composition.

The subject invention further provides “microbe-based products,” which are products that are to be applied in practice to achieve a desired result. The microbe-based product can be simply a microbe-based composition harvested from a microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute or complete prevention, meaning it may still develop at a later time. Prevention can include reducing the severity of the onset of such a situation or occurrence, and/or stalling its development to a more severe or extensive situation or occurrence.

The term “Cyanobacterium” refers to a member from the group of photoautotrophic prokaryotic microorganisms that can utilize solar energy and fix carbon dioxide. Bacterial genera suitable for use according to the current invention, include Synechocystis, Synechococcus, Anabaena, Chroococcidiopsis, Cyanothece, Lyngbya. Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, Tolypothrix and Scytonema.

The term “algae” refers to a member from the group of photoautotrophic eukaryotic microorganisms that can utilize solar energy and fix carbon dioxide. Algal genera suitable for use according to the current invention, include Chlorella, Chlamydomonas, Dunaliella, Bracteacoccus, Prasinoderma, and Nannochloropsis.

As used herein, a “biofilm” is a complex aggregate of microorganisms, such as bacteria, wherein the cells adhere to each other and/or to a surface. The cells in biofilms are physiologically distinct from planktonic cells of the same organism, which are single cells that can float or swim in liquid medium.

As used herein, the phrase “carbon mineralization” or “greenhouse gas mineralization” refers to a process of removing carbon dioxide or other greenhouse gases from the atmosphere and storing the components of carbon dioxide or other greenhouse gases, including carbon, in solid forms in or on the Earth, preferably as a means for carbon sequestration.

As used herein, “nutraceuticals” refer to biologically beneficial compositions that are consumed by humans and/or animals including, for example, drugs, dietary supplements, food ingredients, or foods.

As used herein, a “soil amendment” or a “soil conditioner” is any compound, material, or combination of compounds or materials that are added into soil to enhance the properties of the soil. Soil amendments can include organic and inorganic matter. Nutrient-rich, moist, low saline, microbially-rich, appropriate SOC, well-draining soil is essential for the growth and health of plants, and thus, soil amendments can be used for enhancing the plant biomass by altering the nutrient, salinity, microbial content SOC content, and moisture content of soil. Soil amendments can also be used for improving many different qualities of soil, including but not limited to, soil structure (e.g., aggregate content and preventing compaction); improving the nutrient concentration and storage capabilities; improving water retention in dry soils; and improving drainage in waterlogged soils.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” refers to a negative alteration, and the term “increase” refers to a positive alteration, wherein the negative or positive alteration is at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

As used herein, “reference” refers to a standard or control condition.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference in their entirety.

### Microbe-Based Compositions

In certain embodiments, the subject invention provides a microbe-based composition for reducing atmospheric GHGs, wherein the composition comprises one or more beneficial microorganisms.

Advantageously, in preferred embodiments, the subject compositions can convert GHGs to mineral precipitates. For example, the compositions can convert carbon dioxide in the atmosphere or in emission streams to calcium carbonate, magnesium carbonate, or a bicarbonate, such as, for example, sodium bicarbonate. In some embodiments, the subject compositions can also reduce carbon dioxide concentrations in the atmosphere. In some embodiments, the composition can also enhance the growth and health of and plants and animals through the use of the mineral compounds and/or microorganisms as, for example, soil amendments, nutraceuticals, and feed additives. Furthermore, the compounds resulting from microbial growth can enhance carbon sequestration when applied to surface and/or underground repositories, thereby producing carbon sinks for the sequestration of carbon.

In preferred embodiments, the beneficial microorganisms of the subject compositions are algae and/or cyanobacteria. The microorganisms in the composition may be in an active or inactive form, or in the form of vegetative cells, spores, heterocysts or any other form. The composition may also contain a combination of any of these microbial forms.

The microorganisms and/or microbial growth by-products of the subject compositions can be obtained through cultivation processes ranging from small to large scale. These cultivation processes include, but are not limited to, open pond cultivation, including, for example, troughs or round or raceway ponds. The ponds can be outdoor or indoor ponds or troughs.

In certain embodiments, the compositions of the subject invention can comprise the growth medium in which the beneficial microorganism and/or the growth by-product was produced. The microorganisms of the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a bacterium, particularly a cyanobacteria. Bacterial genera suitable for use according to the current invention include Synechocystis, Synechococcus, Anabaena, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, Tolypothrix, and Scytonema.

In one embodiment, the microorganism is an alga. Algal genera suitable for use according to the current invention include Chlorella, Chlamydomonas, Dunaliella, Bracteacoccus, Prasinoderma, and Nannochloropsis.

In one specific embodiment, the composition comprises about 1×10⁶ to about 1×10¹³, about 1×10⁷ to about 1×10¹², about 1×10⁸ to about 1×10¹¹, or about 1×10⁹ to about 1×10¹⁰ CFU/ml of each species of microorganism present in the composition. In one embodiment, the composition comprises about 1% to about 100% microorganisms total by volume, about 10% to about 90%, or about 20% to about 75%.

In certain specific embodiments, the composition comprises one or more bacteria, algae, and/or one or more growth by-products thereof. In one embodiment, the composition comprises one or more mineral compounds synthesized by a microorganism, including, for example, calcium carbonate, magnesium carbonate, or a bicarbonate, such as, for example, sodium bicarbonate. In certain preferred embodiments, the mineral compounds can be synthesized by the bacteria and/or algae.

In one embodiment, the growth by-product can be purified from the growth medium in which it was produced. Alternatively, in one embodiment, the growth by-product is utilized in crude form. The crude form can comprise, for example, a liquid supernatant resulting from cultivation of a microbe that produces the growth by-product of interest, including residual cells and/or nutrients.

The growth by-products can include metabolites or other biochemicals produced as a result of cell growth, including, for example, minerals, amino acids, peptides, proteins, enzymes, biosurfactants, solvents and/or other metabolites. In preferred embodiments, the metabolite is a mineral compound, such as, for example, calcium carbonate, magnesium carbonate, or a bicarbonate, such as, for example, sodium bicarbonate.

In one embodiment, the subject composition can comprise one or more additional substances and/or nutrients to supplement the nutritional needs of the human, the animal and/or of the plants in the field or pasture, such as, for example, sources of amino acids (including essential amino acids), peptides, proteins, vitamins, microelements, fats, fatty acids, lipids, carbohydrates, sterols, enzymes, and minerals such as calcium, magnesium, phosphorus, potassium, sodium, chlorine, sulfur, chromium, cobalt, copper, iodine, iron, manganese, molybdenum, nickel, selenium, and zinc. In some embodiments, the microorganisms of the composition produce and/or provide these substances.

In one embodiment, the mineral compound has been purified from the growth medium in which it was produced. Alternatively, in one embodiment, the mineral compound is utilized in crude form comprising growth broth resulting from cultivation of a mineral-producing microbe. This crude form mineral solution can comprise from about 0.001% to 99%, from about 0.01% to about 90%, from about 0.1% to about 85%, from about 1% to about 75%, from about 25% to about 75%, from about 30% to about 70%, from about 35% to about 65%, from about 40% to about 60%, from about 45% to about 55%, or about 50% pure mineral, along with residual cells and/or nutrients.

In one embodiment, the composition can further comprise water. For example, the microorganism and/or growth by-products can be mixed with water and administered to the animal. In another embodiment, the composition can be mixed with an animal's or human's drinking water as, for example, a feed additive and/or supplement. The drinking water composition can comprise, for example, 0.01 g/L to about 100 g/L, about 0.1 g/L to about 50 g/L, 1 g/L to about 50 g/L of the composition, about 2 g/L to about 20 g/L, or about 5 g/L to about 10 g/L.

In certain embodiments, the composition comprises a carrier that is suitable for oral delivery of the composition to the gastrointestinal tract of an animal or human. Carriers can be comprised of solid-based, dry materials for formulation into tablet, capsule or powdered form; or the carrier can be comprised of liquid or gel-based materials for formulations into liquid or gel forms.

In one embodiment, the composition can further comprise pre-made wet or dry animal feed, wherein the pre-made food has been cooked and/or processed to be ready for animal consumption. For example, the microorganism and/or growth by-products can be poured onto and/or mixed with the pre-made food, or the microorganism and/or growth by-products can serve as a coating on the outside of dry animal food pieces, e.g., morsels, kibbles or pellets.

In one embodiment, the composition can further comprise raw ingredients for making animal feed, wherein the raw ingredients, together with the microorganism and/or growth by-products, are then cooked and/or processed to make an enhanced dry or wet feed product.

The composition can be added to the wet or day feed and/or raw feed ingredients at a concentration of, for example, about 0.01% to about 99.9%, about 0.1% to about 99%, about 1% to about 75%, or about 5% to about 50% by weight.

As used herein, “dry food” refers to food that contains a limited moisture content, typically in the range of about 5% to about 15% or 20% w/v. Typically, dry processed food comes in the form of small to medium sized individual pieces, e.g., morsels, kibbles, treats, biscuits, nuts, cakes or pellets.

In one embodiment, the composition can further comprise raw ingredients for making animal feed, wherein the raw ingredients, together with the microorganism and/or growth by-products, are then cooked and/or processed to make an enhanced dry or wet feed product. Raw ingredients can include, for example, grains, grasses, roughage, forage, hay, straw, seeds, nuts, crop residue, vegetables, fruits, dried plant matter, and other flavorings, additives and/or sources of nutrients. In one embodiment, the composition is added to the raw food ingredients at a concentration of about 0.1% to about 50%, about 1% to about 25%, or about 5% to about 15% by weight.

The supplemented dry food pieces can comprise consistent concentrations of the composition per piece. In another embodiment, the composition can be utilized as a surface coating on the dry food pieces. Methods known in the art for producing dry processed foods can be used, including pressurized milling, extrusion, and/or pelleting.

In an exemplary embodiment, dry food may be prepared by, e.g., screw extrusion, which includes cooking, shaping and cutting raw ingredients into a specific shape and size in a very short period of time. The ingredients may be mixed into homogenous expandable dough and cooked in an extruder and forced through a die under pressure and high heat. After cooking, the pellets are then allowed to cool, before optionally being sprayed with a coating. This coating may comprise, for example, liquid fat or digest, including liquid or powdered hydrolyzed forms of an animal tissue such as liver or intestine from, e.g., chicken or rabbit, and/or a nutritional oil. In other embodiments, the pellet is coated using a vacuum enrobing technique, wherein the pellet is subjected to vacuum and then exposed to coating materials after which the release of the vacuum drives the coating materials inside the pellet. Hot air drying can then be employed to reduce the total moisture content to 10% or less.

In one embodiment, the dry food is produced using a “cold” pelleting process, or a process that does not use high heat or steam. The process can use, for example, liquid binders with viscous and cohesive properties to hold the ingredients together without risk of denaturing or degrading important components and/or nutrients in the compositions of the subject invention.

In one embodiment, the composition can be applied to animal fodder, or cut and dried plant matter, such as hay, straw, silage, sprouted grains, legumes and/or grains.

In one embodiment, the composition may be prepared as a spray-dried biomass product. The biomass may be separated by known methods, such as centrifugation, filtration, separation, decanting, a combination of separation and decanting, ultrafiltration or microfiltration.

In one embodiment, the subject composition can comprise additional nutrients to supplement an animal's diet and/or promote health and/or well-being in the animal, such as, for example, sources of amino acids (including essential amino acids), peptides, proteins, vitamins, microelements, fats, fatty acids, lipids, carbohydrates, sterols, enzymes, prebiotics, and trace minerals such as, iron, copper, zinc, manganese, cobalt, iodine, selenium, molybdenum, nickel, fluorine, vanadium, tin and silicon.

In some embodiments, the subject composition can also promote plant health and growth for the plants in a field or pasture. In one embodiment, the subject compositions are compatible for use with agricultural compounds characterized as fertilizers, such as, e.g., N-P-K fertilizers, calcium ammonium nitrate 17-0-0, potassium thiosulfate, nitrogen (e.g., 10-34-0, Kugler KQ-XRN, Kugler KS-178C, Kugler KS-2075, Kugler LS 6-24-6S, UN 28, UN 32), and/or potassium; fungicides, such as, for example, chlorothalonil, manicozeb hexamethylenetetramine, aluminum tris, azoxystrobin, Bacillus spp. (e.g., B. licheniformis strain 3086, B. subtilis, B. subtilis strain QST 713), benomyl, boscalid, pyraclostrobin, captan, carboxin, chloroneb, chlorothalonil, copper sulfate, cyazofamid, dicloran, dimethomorph, etridiazole, thiophanate-methyl, fenamidone, fenarimol, fludioxonil, fluopicolide, flutolanil, iprodione, mancozeb, maneb, mefanoxam, fludioxonil, mefenoxam, metalaxyl, myclobutanil, oxathiapiprolin, pentachloronitrobenzene (quintozene), phosphorus acid, propamocarb, propanil, pyraclostrobin, Reynoutria sachalinensis, Streptomyces spp. (e.g., S. griseoviridis strain K61, S. lydicus WYEC 108), sulfur, urea, thiabendazole, thiophanate methyl, thiram, triadimefon, triadimenol, and/or vinclozolin; growth regulators, such as, e.g., ancymidol, chlormequat chloride, diaminozide, paclobutrazol, and/or uniconazole; herbicides, such as, e.g., glyphosate, oxyfluorfen, and/or pendimethalin; insecticides, such as, e.g., acephate, azadirachtin, B. thuringiensis (e.g., subsp. israelensis strain AM 65-52), Beauveria bassiana (e.g., strain GHA), carbaryl, chlorpyrifos, cyantraniliprole, cyromazine, dicofol, diazinon, dinotefuran, imidacloprid, Isaria fumosorosae (e.g., Apopka strain 97), lindane, and/or malathion; adjuvants; surfactants; water treatments, such as, for example, glycolipids, lipopeptides, deet, diatomaceous earth, citronella, essential oils, mineral oils, garlic extract, chili extract, and/or any known commercial and/or homemade pesticide that is determined to be compatible by the skilled artisan having the benefit of the subject disclosure.

In some embodiments, the subject composition can also promote human health, particularly in the form of a nutraceutical.

Production of Microorganisms and/or Microbial Growth By-Products

The subject invention utilizes methods for cultivation of microorganisms. The subject invention further utilizes cultivation processes that are suitable for cultivation of microorganisms on a desired scale. These cultivation processes include, but are not limited to, open pond cultivation, including, for example, round or raceway ponds or, preferably, a trough production system. The ponds or troughs can be located outdoor or indoor.

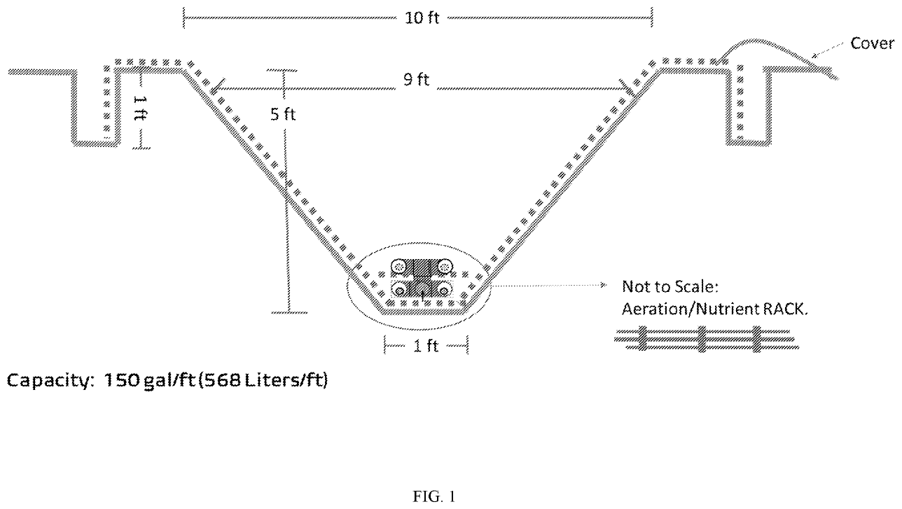

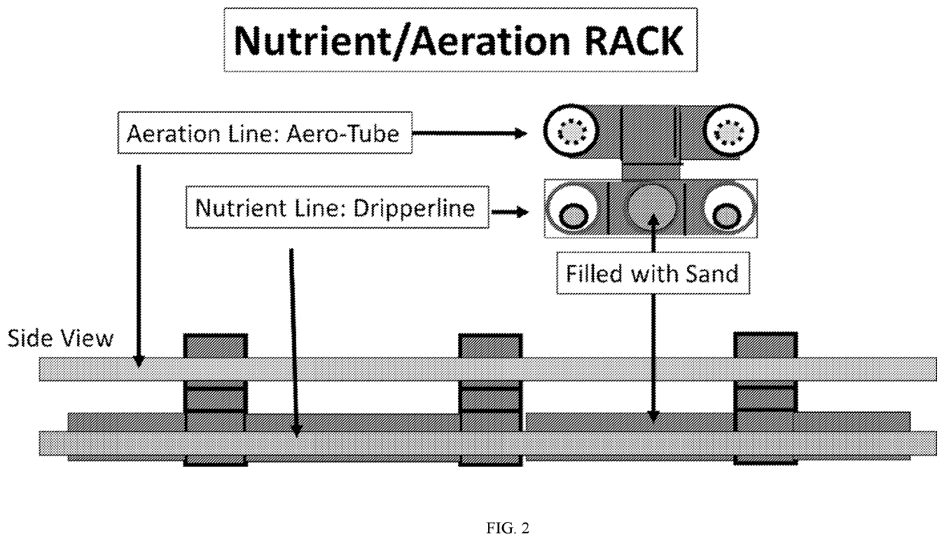

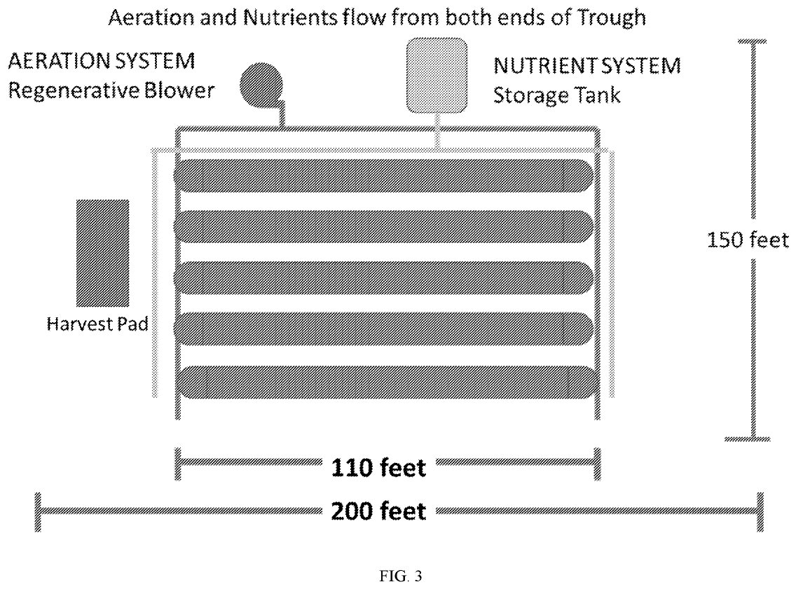

In certain embodiments, the cyanobacteria and/or algae can be grown in a trough production system (FIG. 1). In certain embodiments, the trough can be in the shape of a “V” as opposed to common flat-bottomed raceway growth systems. In certain embodiments, the trough can be at least about 1 foot, about 2, about 3, about 3.5, about 4, about 4.5, about 5, about 5.5, about 6, about 6.5, about 7, about 8, about 9, about 10, or more feet deep at the center of the “V”. In certain embodiments, the trough can be about 1 foot to about 10 feet, about 2 feet to about 9 feet, 3 feet to about 8 feet, about 4 feet to about 7 feet, or about 4 feet to about 5 feet deep at the center of the “V”. In certain embodiments, the trough can be about 1, about 2, about 3, about 4, about 5, about 6, about 7, about 8, about 9, about 10, about 11, about 12, about 13, about 14, about 15, about 16, about 17, about 18, about 19, about 20, or more feet wide at its widest point (e.g., when measured at the top of the trough). In certain embodiments, the trough can be about 1 foot to about 20 feet, about 2 feet to about 19 feet, 3 feet to about 18 feet, about 4 feet to about 17 feet, or about 5 feet to about 15 feet wide at its widest point (e.g., when measured at the top of the trough). In certain embodiments, the trough can be at least about 1 inch, about 2 inches, about 3 inches, about 4 inches, about 5 inches, about 6 inches, about 7 inches, about 8 inches, about 9 inches, about 10 inches, about 11 inches, about 1 foot, about 2 feet, about 3 feet, about 4 feet, about 5 feet, or more feet wide at its narrowest point (e.g., when measured at the base of the trough). In certain embodiments, the trough can be about 1 inch to about 5 feet, about 2 inches to about 4 feet, 3 inches to about 3 feet, about 4 inches to about 2 feet, or about 6 inches to about 1.5 feet wide at its narrowest point (e.g., when measured at the base of the trough). In certain embodiments, the angle of the side walls of the V-shaped trough are greater than 0° and less than 90° or at least about 0.01° to about 89.99°, about 0.1° to about 89.9°, about 1° to about 89°, about 10° to about 80°, about 20° to about 70°, or about 45°.

In certain embodiments, the trough can be at least about 10 feet, about 25 feet, about 50 feet, about 75 feet, about 90 feet, about 100 feet, about 110 feet, about 125 feet, about 150 feet, about 200 feet, about 300 feet, about 400 feet, about 500 feet, about 550 feet, about 600 feet, about 700 feet, about 800 feet, about 900 feet, about 1000 feet, about 1500 feet, about 2000 feet, or more feet long. In certain embodiments, the trough can hold about 1, about 2, about 3, about 4, about 5, about 6, about 7, about 7.48, about 8, about 9, about 10, about 11, about 12, about 13, about 14, about 15, or more gallons of per cubic foot. In certain embodiments, the trough can hold about 10, about 25, about 50, about 75, about 100, about 125, about 150, about 175, about 200, about 225, about 250, about 300, about 350, about 400, about 450, about 500, or more gallons per linear foot. In certain embodiments, the trough can be covered entirely or partially by, for example, a plastic liner.

In certain embodiments, a series of 1, 2, 3, 4, 5, 6, 7, 8, 9, 10, 11, 12, 13, 14, 15, 16, 17, 18, 19, 20, about 25, about 50, about 75, about 100, or more troughs can be used in a growth system. In certain embodiments, the microorganisms, nutrients, growth by-products, and/or growth media can cycle through the troughs, or the microorganisms, nutrients, growth by-products, and/or growth media can remain in a single trough or a portion (e.g., about 50%) of the troughs in the system. In preferred embodiments, the growth system can comprise about five 110 ft long troughs. In certain embodiments, the capacity of the trough system can be about 10,000 gallons, about 20,000 gallons, about 30,000 gallons, about 40,000 gallons, about 50,000 gallons, about 60,000 gallons, about 70,000 gallons, about 80,000 gallons, about 90,000 gallons, about 100,000 gallons, about 110,000 gallons, about 120,000 gallons, about 130,000 gallons, about 140,000 gallons, about 150,000 gallons, about 160,000 gallons, or more gallons. In certain embodiments, the capacity of the trough system can be about 10 gallons to about 10,000 gallons, about 50 gallons to about 5,000 gallons, about 100 gallons to about 1,000 gallons, or about 150 gallons per foot of length of the trough.

In certain embodiments 1, 2, 3, 4, 5, 6, 7, 8, 9, 10 or more trenches can run parallel to a portion of the trough or the entire length of the trough. In certain embodiments, the trench or trenches can be at least about 1 inch, about 2 inches, about 3 inches, about 4 inches, about 5 inches, about 6 inches, about 7 inches, about 8 inches, about 9 inches, about 10 inches, about 11 inches, about 1 foot, about 2 feet, about 3 feet, about 4 feet, about 5 feet, or more feet deep. In certain embodiments, the trench can be about 1 inch to about 5 feet, about 2 inches to about 4 feet, 3 inches to about 3 feet, about 4 inches to about 2 feet, or about 6 inches to about 1.5 feet deep. In certain embodiments, the trench can be at least about 1 inch, about 2 inches, about 3 inches, about 4 inches, about 5 inches, about 6 inches, about 7 inches, about 8 inches, about 9 inches, about 10 inches, about 11 inches, about 1 foot, about 2 feet, or more feet wide. In certain embodiments, the trench can be about 1 inch to about 5 feet, about 2 inches to about 4 feet, or about 3 inches to about 3 feet wide. In preferred embodiments, one trench runs parallel to the trough on each side of the trough. In certain embodiments, the trench can be covered entirely or partially by, for example, a plastic liner.

In certain embodiments, the trough and/or trench can be covered such that the trough and/or trench is substantially or entirely closed from the outside environments. The cover can be translucent or transparent in order to allow light to penetrate to the trough, including covers made from, for example, polyethylene, polypropylene, vinyl, acrylic, polycarbonate, polyester, nylon, polyvinyl chloride, cellulose acetate, or cellophane. The cover can be self-supporting; alternatively, a frame can support the cover. In certain embodiments, the frame can extend from one side of the trough and/or trench to the other side in the shape of a square, hoop, rectangle, trapezoid, or other shape that enables both coverage of the trough and/or trench and substantial or complete segregation of the trough and/or trench from the outside environment. The cover can also reduce or eliminate evaporation of the water, growth media, or gases, including carbon dioxide, from the trough, reduce or eliminate microbial contamination of the trough, and moderate the temperature of the trough.

In certain embodiments, the trough can contain a nutrient/aeration rack that can provide the mixing action and uniform nutrient distribution. In certain embodiments, the nutrient/aeration rack can be at least about 1 inch, about 2 inches, about 3 inches, about 4 inches, about 5 inches, about 6 inches, about 7 inches, about 8 inches, about 9 inches, about 10 inches, about 11 inches, about 1 foot, about 1.5 feet, about 2 feet, about 3 feet, about 4 feet, about 5 feet, or more feet wide. In certain embodiments, the nutrient/aeration rack can be about 1 inch to about 5 feet, about 2 inches to about 4 feet, 3 inches to about 3 feet, about 4 inches to about 2 feet, or about 6 inches to about 1.5 feet long. The rack can be designed to be removable yet be of sufficient weight to stay on the bottom of the trough. In certain embodiments, the nutrient/aeration rack can combine aeration and motive force to the cyanobacteria and/or algae growing within the trough production system to distribute nutrients uniformly. In certain embodiments, the nutrient/aeration rack can move microorganisms, nutrients, growth by-product, growth media from the bottom of the trough up each side of the trough, and, optionally, into and/or out of a trench running parallel to the trough.

In certain embodiments, the trough can be aerated by an aeration system. In certain embodiments, the aeration system comprises a pressurized air system. The pressurized air can be created using, for example, a regenerative blower or an air compressor. In certain embodiments, the aeration system can be connected to the trough at 1, 2, 3, 4, 5, 6, 7, 8, or more locations within a single trough. In certain embodiments, the aeration system can be connected to the nutrient/aeration rack within the trough. In certain embodiments, the air can be delivered at a rate of about 0.1 to about 100, about 0.2 to about 50, about 0.3 to about 25, about 0.4 to about 20, about 0.5 to about 15, about 0.6 to about 10, about 0.7 to about 5, or about 0.8 cubic feet per minute per foot (CFM/ft).

In certain embodiments, nutrients can be provided to the microorganisms in a trough using nutrient supply lines. In certain embodiments, the nutrient supply lines can provide nutrients carried by or dissolved in water. The water-based nutrient solution can be delivered to the trough at a rate of about 0.1 to about 100, about 0.2 to about 50, about 0.3 to about 25, about 0.4 to about 20, about 0.5 to about 15, about 0.6 to about 15, about 0.7 to about 15, about 1 to about 12, or about 10 gallons per minute (GPM). The pressurized nutrients can be generated using, for example, a water pump. In certain embodiments, the aeration system can be connected to the trough at 1, 2, 3, 4, 5, 6, 7, 8, 9, 10, 11, 12, 13, 14, 15, 16, 17, 18, 19, 20 or more locations per about every 10 feet of trough length within a single trough. In certain embodiments, the nutrient supply lines can be connected to the nutrient/aeration rack within the trough. In certain embodiments, the nutrients supply can be delivered by an injector, such as, for example, a venturi injector.

In certain embodiments, the trough can be connected (directly or indirectly) to an emission and/or effluent stream from an industrial source. The industrial source can provide carbon dioxide or other nutrient sources. In preferred embodiments, the carbon dioxide is at a concentration higher than the concentration of carbon dioxide in the atmosphere, such as, for example, at least about 0.04%, to about 25%, about 0.5% to about 14%, about 0.75% to about 10%, or about 1% to about 5%. Sources of emissions or effluents can include power generation stations, particularly those that generate electricity using coal, oil, natural gas, methane, or a combination thereof; industrial production plants, such as, iron and steel production plants, automobile manufacturing plants, petroleum refineries, cement production plants, glass production plants, food production plants, and ethanol or other biofuel production plants, including, for example, biogas, biodiesel, methanol, butanol, and wood; and landfills. In certain embodiments, the methods can comprise the use of existing CO₂ scrubber systems for removal and/or storage of carbon dioxide from emission sources. The scrubbed CO₂ can be used as a carbon source for the growth of microorganisms. In certain embodiments, the methods can be used in conjunction with scrubber systems, either before the emissions are scrubbed, after the emissions are scrubbed, or both before and after the emissions are scrubbed.

The microbe growth vessel used according to the subject invention can also be any closed system, including, for example, a cultivation reactor for industrial or small-scale use. In one embodiment, the vessel may have functional controls/sensors or may be connected to functional controls/sensors to measure important factors in the cultivation process, such as pH, oxygen, carbon dioxide concentration, pressure, temperature, humidity, light intensity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, atmospheric nitrogen (N₂), sodium nitrate, cobalt nitrate, potassium nitrate, ammonium nitrate, ammonium sulfate, ammonium phosphate, ammonia, urea, and/or ammonium chloride. These nitrogen sources may be used independently or in a combination of two or more.

The method can further comprise supplementing the cultivation with a carbon source. The carbon source can be carbon dioxide; a carbohydrate, such as arabinose, glucose, sucrose, lactose, fructose, trehalose, mannose, mannitol, and/or maltose. These carbon sources may be used independently or in a combination of two or more. In preferred embodiments, the carbon source can be an emission stream from, for example, a power generation station, industrial production plants, or landfills. In certain embodiments, humates and/or kelps can be used as supplements.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, copper sulfate, calcium chloride, sodium chloride, calcium carbonate, and/or sodium carbonate. These inorganic salts may be used independently or in a combination of two or more.

In one embodiment, specific nutrients are added to and/or applied concurrently with the microbe-based product to enhance microbial inoculation and growth. These can include, for example, nitrates, sulfates, potassium, calcium, sodium, magnesium, sulfur, boron, iron, manganese, molybdenum, copper, cobalt, and/or zinc. The nutrients can be derived from, for example, sodium nitrate, dipotassium phosphate, magnesium sulfate, calcium chloride, citric acid, ferric ammonium citrate, EDTA disodium salt, sodium carbonate, boric acid, manganese chloride, zinc sulfate, sodium molybdate, copper sulfate, and/or cobalt nitrate.

In certain embodiments, the growth medium is BG-11 (see Allen M M, Stanier R Y. Selective isolation of blue-green algae from water and soil. Microbiology. 1968; 51:203-9.) or Bold's Basal Medium (Brown R M Jr, Larson D A, Bold H C. Airborne Algae: Their Abundance and Heterogeneity. Science. 1964 Feb. 7; 143(3606):583-5 and Nichols H W and Bold H C. (1965), Trichosarcina polymorpha Gen. et Sp. November Journal of Phycology, 1:34-38.).

In certain embodiments, the cyanobacteria and/or algae are exposed to a light intensity of at least about 1000 lux, about 1000 lux to about 120,000 lux (e.g., bright sunlight), about 2,000 lux to about 5,000 lux, or about 2,000 lux to about 3,000 lux. In certain embodiments, the cyanobacteria are exposed to the light for at least about 6 hours, about 8 hours, about 12 hours, about 18 hours, or about 24 hours per day.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state.

The pH of the culture should be suitable for the microorganism of interest as well as for the soil environment to which the composition will be applied. In some embodiments, the pH is about 7.0 to about 12.0, about 7.0 to about 11.5, about 7.0 to about 11.0, about 7.0 to about 10.5, about 7.0 to about 10.0, about 7.0 to about 9.5, about 7.0 to about 9.0, about 8.0 to about 7.5, about 8.0 to about 7.5, about 8.0 to about 10.0, about 8.0 to about 9.5, or about 8.0 to about 9.0. Buffers, and pH regulators, such as carbonates and phosphates, may be used to stabilize pH near a preferred value.

In one embodiment, the method of cultivation is carried out at about 5° to about 100° C., about 15° to about 60° C., about 20° to about 50° C., about 20° to about 45° C., about 25° to about 40° C., about 25° to about 37° C., about 25° to about 35° C., about 30° to about 35° C., or about 30° C. In one embodiment, the cultivation may be carried out continuously at a constant temperature. In another embodiment, the cultivation may be subject to changing temperatures.

In one embodiment, the equipment used in the method and cultivation process is sterile. The cultivation equipment such as the pond/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the growth medium may be sterilized.

In one embodiment, the subject invention further provides a method for producing microbial metabolites such as, for example, mineral precipitates, by cultivating a microbe strain of the subject invention under conditions appropriate for growth and metabolite production. The metabolite content produced by the method can be, for example, at least 0.0001%, 0.001%, 0.01%, 0.1%, 1%, 5%, 10%, or 20%.

The microbial growth by-product produced by microorganisms of interest may be retained in the microorganisms or secreted into the growth medium. The medium may contain compounds that stabilize the activity of microbial growth by-product.

The cell concentration may be, for example, at least 1×10⁶ to 1×10¹³, 1×10⁷ to 1×10¹², 1×10⁸ to 1×10¹¹, or 1×10⁹ to 1×10¹⁰ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the growth product is removed at any one time. In this embodiment, biomass with viable cells, spores, or heterocysts remains in the vessel as an inoculant for a new cultivation batch. The composition that is removed can contain cells, spores, heterocysts, or any combination of thereof. In this manner, a quasi-continuous system is created.

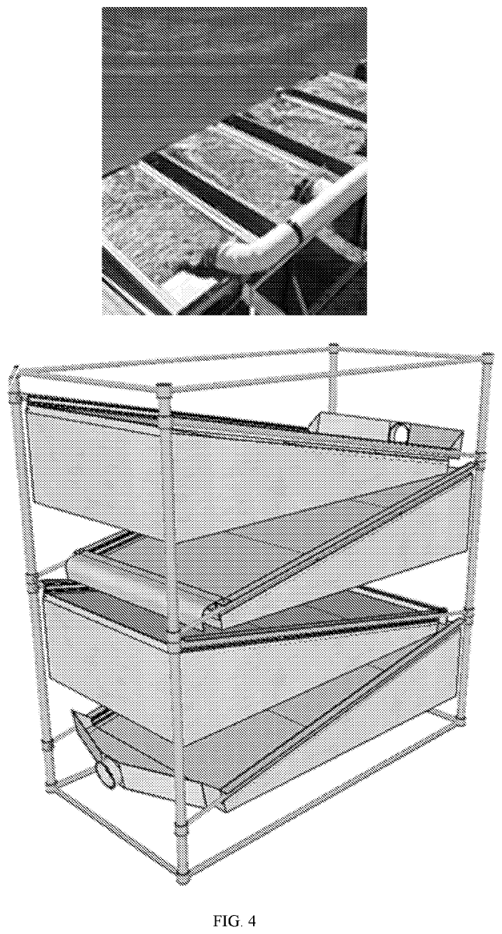

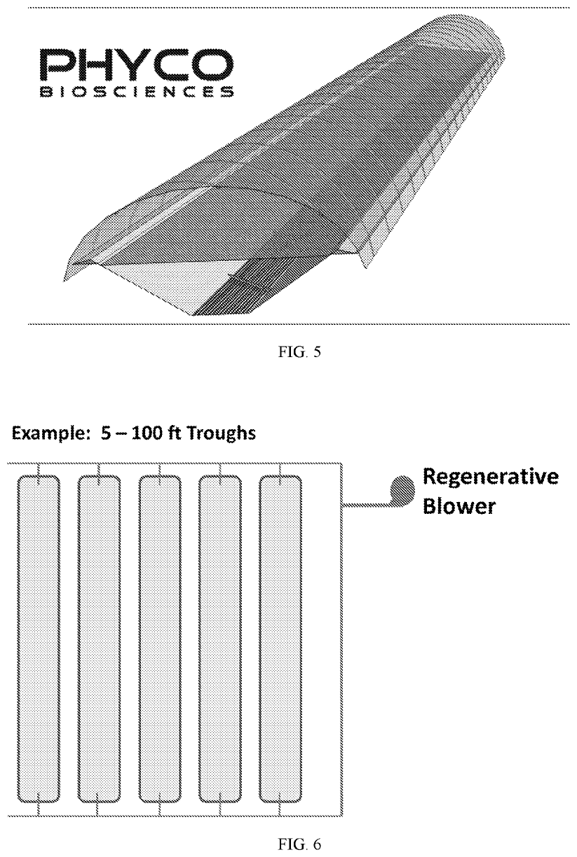

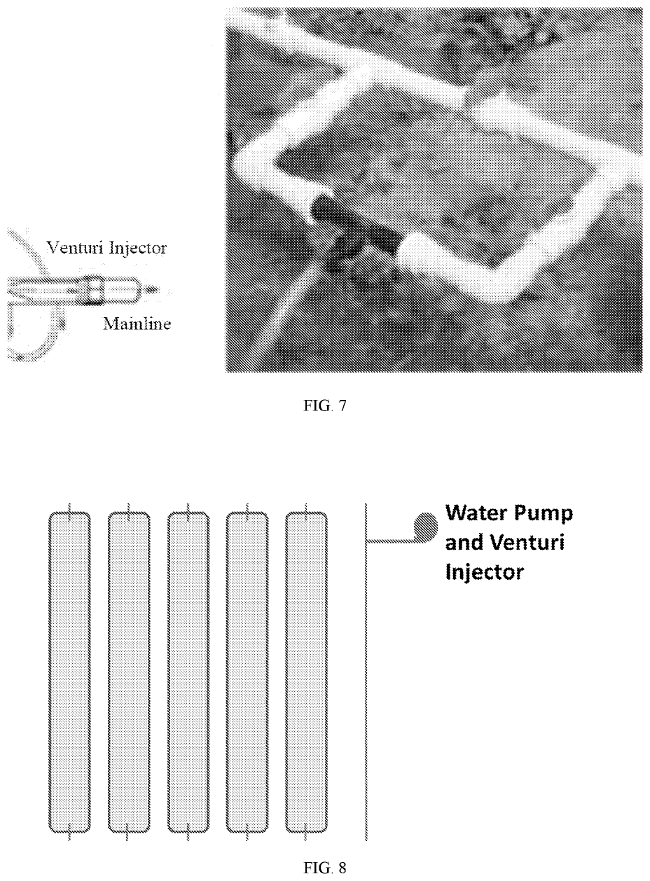

In certain embodiments, a sloped screen concentrator can be used for harvesting a microorganism (FIG. 4). In certain embodiments, the sloped screen concentrator comprises a series of screens that are angled to permit gravity to move microorganisms of at least 10 microns in size across the top of the screens, which can be collected or removed from the system, while microorganisms and other compounds smaller than 10 microns in size flow through the screens and back into the trough system. In certain embodiments, the sloped screen concentrator can be located adjacent to the trough system. The contents of the trough and/or trench can be pumped to the top of the sloped screen harvester. In certain embodiments, the sloped screen concentrator can concentrate the pumped liquid from the trough system by a factor of about 1.5-times to about 5-times, about 2-times to about 4-times, or about 2.5-times. In certain embodiments, the concentrated liquid can be used for directly nutraceuticals, animal feed, and/or soil amendment or can be processed further to purify the cells and/or grow by-products, including for example, synthesized minerals, such as, for example, calcium carbonate, magnesium carbonate, or a bicarbonate, such as, for example, sodium bicarbonate. The minerals can be injected into or applied to repositories for long-term sequestration of carbon.

Advantageously, the method does not require complicated equipment or high energy consumption. The microorganisms of interest can be cultivated at small or large scale on site and utilized, including at sites that contain emission and/or effluent streams comprising carbon dioxide or other greenhouse gases, including with CO₂ scrubber systems. These sites can include power generation stations, particularly those that generate electricity using coal, oil, natural gas, methane, or a combination thereof; industrial production plants, such as, iron and steel production plants, automobile manufacturing plants, petroleum refineries, cement production plants, glass production plants, food production plants, and ethanol or other biofuel production plants; and landfills.

Advantageously, the microbes can be grown in remote locations. The microbe growth facilities may operate off the grid by utilizing, for example, solar, wind and/or hydroelectric power.

### Methods for Reducing Atmospheric Greenhouse Gas Emissions

In preferred embodiments, the subject invention provides a method for reducing emissions of carbon dioxide and/or other deleterious atmospheric gases, wherein a composition comprising one or more beneficial microorganisms in grown. Preferably, the composition is a microbe-based composition of the subject invention.

Advantageously, in preferred embodiments, the methods result in a reduction of GHG in atmosphere, particularly, carbon dioxide by converting the carbon to dioxide into a mineral compound, such as, for example, calcium carbonate, magnesium carbonate, or a bicarbonate, such as, for example, sodium bicarbonate. In some embodiments, the desired reduction is achieved within a relatively short time period, for example, within 1 week, 2 weeks, 3 weeks or 4 weeks of initiating the growth of the microorganism. In some embodiments, the desired reduction is achieved within, for example, 1 month, 2 months, 3 months, 4 months, 5 months or 6 months after employing the subject methods. In some embodiments, the desired reduction is achieved within 1 year, 2 years, 3 years, 4 years, or 5 years after employing the subject methods.

In some embodiments, the methods can further comprise adding materials to enhance the growth of the microorganisms of the subject composition at the time of application (e.g., adding nutrients and/prebiotics). In one embodiment, the nutrient sources can include, for example, sources of nitrates, sulfates, potassium, calcium, sodium, magnesium, sulfur, boron, iron, manganese, molybdenum, copper, cobalt, and/or zinc. The nutrients can be derived from, for example, sodium nitrate, dipotassium phosphate, magnesium sulfate, calcium chloride, citric acid, ferric ammonium citrate, EDTA disodium salt, sodium carbonate, boric acid, manganese chloride, zinc sulfate, sodium molybdate, copper sulfate, and/or cobalt nitrate. The source of nutrients, particularly carbon dioxide, can be emission and/or effluent streams from industrial sources.

The composition can also be used in combination with other crop management systems, including application of pesticides, herbicides, fertilizers, and/or other soil amendments. In preferred embodiments, the other crop management system is environmentally-friendly and not harmful to humans or livestock.

In some embodiments, prior to applying the composition, the method comprises assessing a human, livestock animal, field and/or pasture for local conditions, determining a preferred formulation for the composition (e.g., the type, combination and/or ratios of microorganisms and/or growth by-products) that is customized for the local conditions, and producing the composition with said preferred formulation.

In one embodiment, the composition is applied either as a liquid or a dried product. In one embodiment the composition is broadcast, either in the liquid or dried form, over or into the soil profile of the field or pasture using, for example, an irrigation system or an aerial spreader. Additionally, the composition can be applied using a manual spreader, such as a broadcast spreader, a drop spreader, a handheld spreader, or a handheld sprayer.

In one embodiment, the composition is formulated into supplemental feed, wherein the composition is added to standard raw food ingredients utilized in producing processed wet and/or dry animal feed.

In some embodiments, the compositions described herein can be co-administered with another feed or nutraceutical composition as a dietary supplement. The dietary supplement can have any suitable form such as a gravy, drinking water, beverage, yogurt, powder, granule, paste, suspension, chew, morsel, liquid solution, treat, snack, pellet, pill, capsule, tablet, sachet, or any other suitable delivery form. The dietary supplement can comprise the subject microbe-based compositions, as well as optional compounds such as vitamins, minerals, probiotics, prebiotics, and antioxidants. In some embodiments, the dietary supplement may be admixed with a feed or nutraceutical composition or with water or other diluent prior to administration to the animal or human.

According to the methods of the subject invention, administration of the microbe-based compositions can be performed as part of a dietary regimen, which can span a period ranging from parturition through the adult life of the animal or human. In certain embodiments, the animal or human is young. In some embodiments, the animal or human is a juvenile, adult, or senior animal or human.

In certain embodiments, the methods can also be used for reducing GHG by way of carbon sequestration, wherein a repository is injected with one or more beneficial microorganisms of the microbe-based composition. In preferred embodiments, the one or more beneficial microorganism and/or the synthesized mineral precipitates, including, for example, calcium carbonate, magnesium carbonate, or a bicarbonate, such as, for example, sodium bicarbonate, can be injected into underground geological formations, in which the mineral is trapped permanently. The underground geological storage can be, for example, a basalt formation, shale formation, deep saline formation, depleted oil and gas reservoirs, or unmincable coal seams. Additionally, the synthesized growth by-products and, optionally, microorganisms can be applied to a surface repository in soil and/or released in a body of water, such as, for example, an ocean, sea, lake, pond, river, stream or bay, in order to sequester carbon.

In one embodiment, the result can be, for example, enhanced vegetative carbon utilization can be in the form of, for example, increased above- and below-ground biomass of plants, including, for example, increased foliage volume, increased stem and/or trunk diameter, enhanced root growth and/or density, and/or increased numbers of plants. In one embodiment, the result can be, for example, increased soil sequestration in the form of, for example, increased plant root growth, increased uptake by microorganisms of organic compounds secreted by plants (including secretions from plant roots) and improved microbial colonization of soil.

In some embodiments, the methods of the subject invention can be utilized for reducing carbon credit usage or for selling carbon credits. Thus, in certain embodiments, the subject methods can further comprise conducting measurements to assess the effect of the method on the production of mineral precipitates and/or the sequestration of carbon. These measurements can be conducted according to known methods in the art, specifically, measuring the amount of mineral precipitates produced by microorganisms and/or measuring the amount of mineral precipitates that are injected into repositories or applied to repositories.

Measurements can be conducted at a certain time point after application of the microbe-based composition. In some embodiments, the measurements are conducted after about 1 week or less, 2 weeks or less, 3 weeks or less, 4 weeks or less, 30 days or less, 60 days or less, 90 days or less, 120 days or less, 180 days or less, and/or 1 year or less.

Furthermore, the measurements can be repeated over time. In some embodiments, the measurements are repeated daily, weekly, monthly, bi-monthly, semi-monthly, semi-annually, and/or annually.

Reducing the Carbon Footprint and/or Carbon Intensity

A “carbon footprint” may be defined herein as a measure of the total amount of carbon dioxide (CO₂) and other GHGs emitted directly or indirectly by a human activity or accumulated over the full life cycle of a product or service. As just one example, the carbon footprint of a fossil fuel-based power generating plant that does not sequester carbon emissions may have a larger carbon footprint than a fossil fuel-based power generating plant that does sequester carbon emissions. In certain embodiments, the sequestration can be accomplished according to the subject invention by injecting calcium carbonate, magnesium carbonate, or a bicarbonate, such as, for example, sodium bicarbonate synthesized by cyanobacteria and/or algae into an underground repository.

Carbon footprints can be calculated using a Life Cycle Assessment (LCA) method, the Argonne GREET model, or can be restricted to the immediately attributable emissions from energy use of fossil fuels. An LCA, also known as life cycle analysis, ecobalance, and cradle-to-grave analysis) is the investigation and valuation of the environmental impacts of a given product or service caused or necessitated by its existence. The life cycle concept of the carbon footprint means that it is all-encompassing and includes all possible causes that give rise to carbon emissions. In other words, all direct (on-site, internal) and indirect emissions (off-site, external, embodied, upstream, downstream) need to be taken into account.

Normally, a carbon footprint is expressed as a CO₂ equivalent or in some markets as a carbon intensity (CI) score. Carbon dioxide equivalency is a quantity that describes, for a given mixture and amount of GHG, the amount of CO₂ that would have the same global warming potential (GWP), when measured over a specified timescale (generally, 100 years). Carbon dioxide equivalency thus reflects time-integrated radiative forcing. The carbon dioxide equivalency for a gas is obtained by multiplying the mass and the GWP of the gas. The following units are commonly used:
a) By the UN climate change panel IPCC: billion metric tonnes of CO₂ equivalent (GtCO₂ eq);
b) In industry: million metric tonnes of carbon dioxide equivalents (MMTCDE);
c) For vehicles: g of carbon dioxide equivalents/km (gCDE/km).

For example, the GWP for methane is 21 and for nitrous oxide 310. This means that emissions of 1 million metric tonnes of methane and nitrous oxide respectively is equivalent to emissions of 21 and 310 million metric tonnes of carbon dioxide.

Various methods exist in the art for calculating or estimating carbon footprints and may be employed in the subject invention.

Advantageously, in preferred embodiments, the subject invention can be useful for reducing and/or reversing the carbon footprint of human activity, which includes reducing the carbon footprint and CI score of producing burning fossil fuels (coal, natural gas, and oil), solid waste, trees and wood products, and also as a result of certain chemical reactions, including through the manufacturing of cement.

To determine the amount of carbon sequestered using the claimed methods and/or compositions, the amount of carbon dioxide that would have been released to the atmosphere if not for the claimed invention can be determined. The amount of carbon sequestered can be measured upon carbon capture, by, for example, measuring the amount of mineral precipitates (e.g., calcium carbonate, magnesium carbonate, or a bicarbonate, such as, for example, sodium bicarbonate) produced by the algae and/or cyanobacteria. Additionally, the amount can be measured at the point of injection or application to the repositories, particularly, the underground geological formations.

A “reduced carbon footprint” means a negative alteration in the amount of carbon dioxide and other GHGs emitted per unit time over the full life cycle of producing a product, through and until the product is ultimately consumed by human consumers. The negative alteration in CO₂ and/or other GHG emissions can be, for example, at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

In some embodiments, the term “carbon footprint” is interchangeable herein with the terms “carbon intensity” and “emission intensity.” Emission intensity is the measure of the emission rate of a given GHG relative to the “intensity” of a specific activity or industrial process (e.g., burning of fuel or the production of cement). The emissions intensity can include emission amount relative to, for example, amount of fuel combusted, amount of a commercial product produced, total distance traveled, and/or number of economic units generated.

Emissions intensity is measured across the entire life cycle of a product. For example, the emissions intensity of fuels is calculated by compiling all of the GHG emissions emitted along the supply chain for a fuel, including all the emissions emitted in exploration, mining, collecting, producing, transporting, distributing, dispensing and burning the fuel.

In addition to reducing the carbon footprint and/or carbon intensity of the production of energy or goods, transportation, or agriculture, in some embodiments, the subject invention can be used for oversetting carbon used by a producer involved in, e.g., agriculture, transportation, energy generation, optionally, in a system utilizing carbon credits.

Advantageously, the systems of the subject invention can decrease the amount of GHG in the atmosphere and, optionally, can sequester carbon. In particular, the compositions and methods utilized according to the subject invention can help in reducing GHG emissions, such as carbon dioxide, while improving the production of valuable commodities.

### Preparation of Microbe-Based Products

One microbe-based product of the subject invention is simply the growth medium containing the microorganisms and, optionally, the microbial growth by-products produced by the microorganisms and/or any residual nutrients. The product of growth may be used directly without extraction or purification.

The microorganisms in the microbe-based products may be in an active or inactive form, or in the form of vegetative cells, spores, heterocysts, or any other form. The microbe-based products may also contain a combination of any of these forms of a microorganism.

In one embodiment, different species or strains of cyanobacteria or algae can be grown separately and then mixed together to produce the microbe-based product. The microbes can, optionally, be blended with the medium in which they are grown and dried prior to mixing. In certain embodiments, the microbes can be dried during the preparation process. In certain embodiments, the microbes can be grown in an open trough or other growth vessel after being dried. In certain embodiments, the microbes can be mixed with a water-based solution before being grown in an open trough or other growth vessel.

In one embodiment, the different strains are not mixed together, but are applied to an open trough or other growth vessel as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the carbon sequestration activity of the microbial growth.

Upon harvesting the microbe-based composition from the growth vessels, further components can be added as the harvested product is placed into containers or otherwise transported for use or storage. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, sodium biphosphate, can be included in the formulation.

In one embodiment, specific nutrients are added to and/or applied concurrently with the microbe-based product to enhance microbial inoculation and growth. These can include, for example, nitrates, sulfates, potassium, calcium, sodium, magnesium, sulfur, boron, iron, manganese, molybdenum, copper, cobalt, and/or zinc. The nutrients can be derived from, for example, sodium nitrate, dipotassium phosphate, magnesium sulfate, calcium chloride, citric acid, ferric ammonium citrate, EDTA disodium salt, sodium carbonate, boric acid, manganese chloride, zinc sulfate, sodium molybdate, copper sulfate, and/or cobalt nitrate.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### REFERENCES

United States Environmental Protection Agency. (2016). “Climate Change Indicators in the United States.” https://www.epa.gov/sites/production/files/2016-08/documents/climate_indicators_2016.pdf. (“EPA Report 2016”).
United States Environmental Protection Agency. (2016). “Overview of Greenhouse Gases.” Greenhouse Gas Emissions. https://www.epa.gov/ghgemissions/overview-greenhouse-gases. (“Greenhouse Gas Emissions 2016”).

1. A method for reducing an atmospheric greenhouse gas, the method comprising growing a microorganism selected from cyanobacteria and algae and contacting a source of said greenhouse gas with the microorganism, wherein the microorganism mineralizes the atmospheric greenhouse gas.

2. The method of claim 1, wherein the microorganism is selected from Synechocystis spp., Synechococcus spp., Anabaena spp., Chroococcidiopsis spp., Cyanothece spp., Lyngbya spp., Phormidium spp., Nostoc spp., Spirulina spp., Arthrospira spp., Trichodesmium spp., Leptolyngbya spp., Plectonema spp., Myxosarcina spp., Pleurocapsa spp., Oscillatoria spp., Pseudanabaena spp., Geitlerinema spp., Euhalothece spp., Calothrix spp., Tolypothrix spp., Scytonema spp., Chlorella spp., Chlamydomonas spp., Dunaliella spp., Bracteacoccus spp., Prasinoderma spp., and Nannochloropsis spp.

3. The method of claim 2, wherein the microorganism is selected from Nostoc spp., Spirulina spp., Tolypothrix spp., and Anabaena spp.

4. The method of claim 1, wherein the atmospheric greenhouse gas is carbon dioxide.

5. The method of claim 1, wherein the source of the atmospheric greenhouse gas is a power generation station, iron and/or steel production plant, automobile manufacturing plant, petroleum refinery, cement production plant, glass production plant, food production plant, ethanol or other biofuel production plant, or landfill.

6-8. (canceled)

9. The method of claim 81, wherein the method produces a mineral selected from calcium carbonate, magnesium carbonate, and a bicarbonate.

10-13. (canceled)

14. The method of claim 1, comprising injecting the microorganism into an underground geological formation.

15. (canceled)

16. The method of claim 1, further comprising applying the microorganism to soil.

17-20. (canceled)

21. The method of claim 1, further comprising assessing the effect of the method on the production of minerals by the microorganism.

22. The method of claim 1, wherein said method results in a reduction of the number of carbon credits used by an operator and/or an increase in the number of carbon credits available for sale by an operator.

23-26. (canceled)

27. A system comprising:
a V-shaped trough, wherein the V-shaped trough serves as a growth reactor for a microorganism;
a trench; wherein the trench runs parallel to the V-shaped trough; and
a nutrient/aeration rack; wherein the nutrient/aeration rack is at the base of the V-shaped trough and moves the microorganism and a growth media from a base of the trough out of the V-shaped trough and into and/or out of the trench.

28. The system of claim 27, wherein the nutrient/aeration rack is connected to a nutrient source for introducing nutrients and water into the V-shaped trough.

29. The system of claim 28, wherein the nutrient source comprises an atmospheric greenhouse gas from an industrial emission, wherein the industrial emission is from a power generation station, iron and steel production plant, automobile manufacturing plant, petroleum refinery, cement production plant, glass production plant, food production plant, ethanol or other biofuel production plant, or landfill.

30-31. (canceled)

32. The system of claim 27, wherein the nutrient/aeration rack is connected to a pressurized air source for aerating the one or more microorganisms in the V-shaped trough.

33. The system of claim 27, the V-shaped trough is connected to a sloped screen concentrator, wherein the sloped screen concentrator comprises a series of screens that are angled to permit gravity to move microorganisms of at least 10 microns in size across the top of the screens and microorganisms and other compounds smaller than 10 microns in size flow through the screens and back into the V-shaped trough.

34-36. (canceled)

37. A method of producing a microorganism and/or a growth by-product thereof, the method comprising:
introducing water, nutrients, and a microorganism into the system of claim 27 to produce a culture; and
cultivating the culture for an amount of time and under conditions favorable for production of a growth by-product.

38. The method of claim 37, wherein the one or more microorganisms are Synechococcus spp., Anabaena spp., Chroococcidiopsis spp., Cyanothece spp., Lyngbya spp., Phormidium spp., Nostoc spp., Spirulina spp., Arthrospira spp., Trichodesmium spp., Leptolyngbya spp., Plectonema spp., Myxosarcina spp., Pleurocapsa spp., Oscillatoria spp., Pseudanabaena spp., Geitlerinema spp., Euhalothece spp., Calothrix spp., Tolypothrix spp., Scytonema spp., Chlorella spp., Chlamydomonas spp., Dunaliella spp., Bracteacoccus spp., Prasinoderma spp., Nannochloropsis spp., or any combination thereof.

39. The method of claim 37, wherein the growth by-product is a mineral.

40. The method of claim 39, wherein the mineral is calcium carbonate, magnesium carbonate, a bicarbonate, or a combination thereof.

41-42. (canceled)

43. The method of claim 37, wherein the nutrients comprise an atmospheric greenhouse gas from industrial emissions, wherein the industrial emissions are from a power generation station, iron and steel production plant, automobile manufacturing plant, petroleum refinery, cement production plant, glass production plant, food production plant, ethanol or other biofuel production plant, or landfill.

44-45. (canceled)
